(19) United States
(12) Patent Application Publication
(10) Pub. No.: US 2012/0064178 A1
(43) Pub. Date: Mar. 15, 2012

(54) Cell-8 Solution

(76) Inventors: Carolyn Flora Anne Dean, Kihei, HI (US); Jeffrey Wallace Edwards, Bremen, GA (US)

(21) Appl. No.: 13/165,657

(22) Filed: Jun. 21, 2011

Related U.S. Application Data
(60) Provisional application No. 61/403,211, filed on Sep. 13, 2010.
(60) Provisional application No. 61/403,437, filed on Sep. 16, 2010.
(60) Provisional application No. 61/403,432, filed on Sep. 16, 2010.
(60) Provisional application No. 61/462,305, filed on Feb. 1, 2011.
(60) Provisional application No. 61/462,315, filed on Feb. 1, 2011.
(60) Provisional application No. 61/462,320, filed on Feb. 1, 2011.

(51) Int. Cl. A61K 33/14; A61P 35/00

(52) U.S. Cl. 424/678

(57) ABSTRACT

Cell-8 Solution is an intravenous solution introduced into the veins of the patient. Cell-8 Solution provides the cells of the body with highly absorbed minerals as well as new RNA information which changes the relationship of the body to the cancer in question. Treatment with Cell-8 Solution will prevent the body from accepting the existence of cancer cells so that cancer cell replication will no longer occur. Although intravenous Cell-8 Solution is superior, other applications include nebulized, transdermal and oral.

### CROSS-REFERENCE TO RELATED APPLICATIONS

I-Cell: U.S. 61/403,211
Cellionose: U.S. 61/403,437
Super Trihydrogen: U.S. 61/403,432
PicoMinerals: U.S. 61/462,305
iBlock: U.S. 61/462,315
Axeion Water: U.S. 61/462,320

### BACKGROUND OF THE INVENTION

The Cell-8 Solution invention pertains to the field of cellular replication. Cell-8 Solution promotes healthy cell replication and does not allow for the procreation or replication of malignant or non-malignant cancer cells.

Cancer is acquired at the cellular level. Therefore, the best solutions are directed at the cellular level to create a microcellular environment that keeps the cells in physiologic balance.

What are cells missing that sends them on an errant path? Until we know if something specific is missing, we should supply the cells with information in the form of cellular nutrients and hydration.

The current treatments for cancer focus on various combinations of chemotherapeutic drugs in what is called “targeted drug therapies”. There are no new breakthroughs. Even adjusting for age, the percentage of Americans dying from cancer is about the same as it was in 1971 (when President Nixon declared war on cancer).

Targeted drug therapies is a misnomer. Such therapies are not able to precisely target cancer cells. They kill all rapidly replicating cells indiscriminately.

Cell-8 Solution is not chemotherapy. Cell-8 Solution enhances normal healthy cell replication, but not cancer cells. Cancer cells, therefore, undergo apoptosis. Since they are no longer being fed, they cease replicating.

Epigenetics has placed needed emphasis on the impact of the cellular environment on genes and chromosomes. Epigenetics informs us that the cellular “soup” in which our genes swim is as important as the gene base pairs themselves.

We also know that impulses that initiate movement in the body arise from internal electrical transmissions. These microcurrents of electricity that pass along the nerves were first measured in 1966. Minerals are the conductor for these bodily electrical currents—most prominently, calcium and magnesium. (Emphasis supplied - Levine B S, Coburn J W, “Magnesium, the mimic/antagonist of calcium.” N Engl J Med, vol. 310, pp. 1253-1255, 1984.)

Since cells are not controlled by a lock-and-key mechanism but by electrical impulse, then, to influence cellular function, picometer-sized minerals are our definitive focus in Cell-8 Solution.

Physicists have identified the human cellular mineral ion channels as being 400-500 picometers in diameter. Therefore, the best absorption at the cellular level occurs with picometer-sized minerals.

The bulk of Cell-8 Solution is a form of lactated Ringer's. The original solution was invented by a British physiologist, Dr. Sydney Ringer, in a series of experiments from 1882-1885. The currently used lactated Ringer's is a solution that is isotonic with blood and given intravenously.

One liter of lactated Ringer's solution contains:
130 mEq of sodium ion=130 mmol/L
109 mEq of chloride ion=109 mmol/L
28 mEq of lactate=28 mmol/L
4 mEq of potassium ion=4 mmol/L
3 mEq of calcium ion=1.5 mmol/L

Dr. Alexis Hartmann added a buffering agent, lactate, to Dr. Ringer's solution to treat acidosis in children. The standard pH for lactated Ringer's is 6.5, but adding lactate makes it an alkaline solution. Lactated Ringer's is currently used to counteract the acidosis that occurs with acute fluid loss or renal failure.

The minerals used in lactated Ringer's are NaCl (sodium chloride), NaC3H5O3 (sodium lactate), CaCl2 (calcium chloride), MgCl2 (magnesium chloride) and KCl (potassium chloride). The chloride minerals are the perfect choice for influencing cell function because the most important mineral ion channels into the cell are chloride channels.

Lactated Ringer's solution is an extremely safe universal fluid replacement. It is used after blood loss due to bums, surgery or any type of trauma, and for patients in renal failure. It's also a standard IV solution for delivering drugs to patients in hospital. It can be used to treat animals either intravenously or subcutaneously. Lactated Ringer's may be taken orally although it has a bitter taste due to the minerals.

Minerals are essential as co-factors in thousands of metabolic processes in the body. Cell-8 Solution incorporates picometer-sized minerals that move quite freely through the chloride mineral channels of cells. Therefore, these picometer-sized minerals are totally absorbed at the cellular level.

This complete absorption (due to their picometer-size) allows the minerals to enhance healthy cell replication as the metabolic processes proceed flawlessly.

Cell-8 Solution sets the metabolic standard that would no longer include the replication of cancer cells.

Other constituents of Cell-8 Solution include:
Patent-Pending Cellionose
Patent Pending iCell
Patent Pending Axeion Water
Patent Pending Super Trihydrogen
Patent Pending PicoMinerals
Patent Pending iBlock

### BRIEF SUMMARY OF THE INVENTION

Cell-8 Solution is an intravenous solution introduced into the veins of patients. Cell-8 Solution provides the cells of the body with highly absorbed minerals as well as new RNA information which changes the relationship of the body to the cancer in question. Treatment with Cell-8 Solution will prevent the body from accepting the existence of cancer cells so that they will no longer be able to replicate. Although intravenous Cell-8 Solution is superior, other applications include nebulized, transdermal and oral.

### BRIEF DESCRIPTION OF THE SEVERAL VIEWS OF THE DRAWING

Drawings will be provided for individual formulas, specifically for unique amino acids.

### DETAILED DESCRIPTION OF THE INVENTION

Cell-8 Solution achieves superior cell production which excludes cancer cell replication.

The Base Formula for Cell-8 Solution:

Each 1000 ml of Cell-8 Solution contains:
1. Cellionose USP 50 g
2. Magnesium chloride hexahydrate (100 picometers) 45 mg
3. Sodium chloride (100 picometers) in a range of 300-650 mg
4. Potassium chloride (100 picometers) 30 mg
5. Calcium chloride, dihydrate (100 picometers) 20 mg
6. Sodium lactate, anhydrous 3100 mg
7. Axeion H20 and
8. SuperTrihydrogen (H302) up to 1000 ml
Cellionose: U.S. 61/403,437
Axeion Water: U.S. 61/462,320
Super Trihydrogen: U.S. 61/403,432

1. Cell-8 Solution halts the replication of cancer cells. This outcome is based on the adjustment to the Cell-8 Solution formulary (specific individual formulas will be scheduled and provided).

2. Cell-8 Solution will have far-reaching ramifications as the new RNA information is delivered via polypeptides so that DNA will permanently affect the cell itself, effecting replication.
